(19) United States
(12) Patent Application Publication
(10) Pub. No.: US 2026/0210751 A1
(43) Pub. Date: Jul. 23, 2026

(54) Method and System for Monitoring Fluid Levels in Medical Devices

(71) Applicant: Becton, Dickinson and Company, Franklin Lakes, NJ (US)

(72) Inventor: Zachary Jarrod Traina, Denville, NJ (US)

(21) Appl. No.: 19/140,507

(22) PCT Filed: Dec. 28, 2023
(86) PCT No.: PCT/US23/86127

§ 371 Date: Jun. 18, 2025

Related U.S. Application Data
(60) Provisional application No. 63/478,211, filed on Jan. 3, 2023.

(51) Int. Cl. G01F 23/292; A61M 39/02

(52) U.S. Cl. CPC G01F 23/292; A61M 39/0247; A61M 2039/0258; A61M 2039/0267; A61M 2039/0288; A61M 2205/3313; A61M 2205/3379

(57) ABSTRACT

A method for monitoring a fluid level in a medical device includes emitting electromagnetic radiation from the needleless connector to the cap, sensing an amount of reflected electromagnetic radiation reflected back toward the needleless connector, transmitting a value of the amount of reflected electromagnetic radiation to a medical device management system, analyzing the value of the amount of reflected electromagnetic radiation to determine if the amount of reflected electromagnetic ration meets a predetermined threshold, and responding based upon the analyzing.

### CROSS-REFERENCE TO RELATED APPLICATION

The present application claims priority to U.S. Provisional Application No. 63/478,211 entitled “Method and System for Monitoring Fluid Levels in Medical Devices” filed Jan. 3, 2023, the entire disclosure of which is hereby incorporated by reference in its entirety.

### BACKGROUND OF THE INVENTION

### Field of the Invention

The present disclosure relates generally to detection of and monitoring fluid levels in medical devices.

### Description of Related Art

Fluid levels in medical devices are typically detected by and monitored with reflective fluid level sensors. Examples of common reflective fluid level detectors include float switches, ultrasonic sensors, capacitive sensors, and similar devices. Drawbacks to current reflective fluid level sensors include need for chemical compatibility, power, cost, and/or size constraints that limit the use of these reflective fluid level detectors.

Accordingly, those skilled in the art continue with research and development efforts in the field of monitoring fluid levels in medical devices.

### SUMMARY OF THE INVENTION

Disclosed is a method for monitoring a fluid level in a medical device.

In one example, the method includes emitting electromagnetic radiation from a first portion of the medical device to a second portion of the medical device, sensing an amount of reflected electromagnetic radiation reflected back toward the first portion of the medical device, transmitting a value of the amount of reflected electromagnetic radiation to a medical device management system, analyzing the value of the amount of reflected electromagnetic radiation to determine if the amount of reflected electromagnetic ration meets a predetermined threshold, and responding based upon the analysis.

Also disclosed is a medical device.

In one example, the medical device includes a needleless connector housing an emitter and a sensor. The medical device further includes a cap removably coupled to the needleless connector, the cap defining a reservoir, the cap comprising a reflector.

Also disclosed is a system for monitoring a fluid level in a medical device.

In one example, the system includes a medical device having a needleless connector including an emitter and a sensor. The medical device further includes a cap removably coupled to the needleless connector, the cap defining a reservoir, the cap housing a reflector having a reflective surface. The system further includes a medical device management system in communication with the medical device.

In accordance with an embodiment of the invention a method for monitoring a fluid level in a medical device includes emitting electromagnetic radiation from the needleless connector toward the cap, sensing an amount of reflected electromagnetic radiation reflected back toward the needleless connector, transmitting a value of the amount of reflected electromagnetic radiation to a medical device management system, analyzing the value of the amount of reflected electromagnetic radiation to determine if the amount of reflected electromagnetic ration meets a predetermined threshold, and responding based upon the analyzing.

In accordance with another embodiment of the present invention, the method further includes coupling a cap to a needleless connector prior to the emitting.

In accordance with another embodiment of the present invention, the method further includes wherein the emitting includes emitting electromagnetic radiation having a wavelength in the range of 800 nm to 1000 nm from an emitter.

In accordance with another embodiment of the present invention, the method further includes wherein, if the value of the amount of reflected electromagnetic radiation meets the predetermined threshold, the responding includes recording environmental conditions present at the time of the sensing.

In accordance with another embodiment of the present invention, the method further includes after a predetermined period of time (T) after the recording, repeating the emitting, sensing, transmitting, analyzing, and responding.

In accordance with another embodiment of the present invention, the method further includes wherein, if the value of the amount of reflected electromagnetic radiation does not meet the predetermined threshold, the responding includes one or more of recording environmental conditions present at the time of the sensing and dispatching an alert.

In accordance with another embodiment of the present invention, the method further includes wherein the needleless connector includes an emitter configured to emit electromagnetic radiation and a sensor, and the cap includes a reflector having a reflective surface, the reflective surface configured to reflect the emitted electromagnetic radiation toward the sensor when the reflective surface is dry.

In accordance with another embodiment of the present invention, the method further includes wherein the amount of reflected electromagnetic radiation correlates to an amount of fluid present in the cap.

In accordance with another embodiment of the present invention, the method further includes wherein the transmitting is performed wirelessly.

In accordance with another embodiment of the present invention, the method further includes wherein the analyzing is performed by a medical device management system.

In accordance with an embodiment of the present invention, a medical device includes a needleless connector housing an emitter and a sensor, and a cap removably coupled to the needleless connector, the cap defining a reservoir, the cap including a reflector.

In accordance with another embodiment of the present invention, the medical device further includes wherein the sensor is in communication with a medical device management system.

In accordance with another embodiment of the present invention, the medical device further includes wherein the emitter includes one or more sources of electromagnetic radiation configured to emit electromagnetic radiation having a wavelength in the range of 800 nm to 1000 nm.

In accordance with another embodiment of the present invention, the medical device further includes wherein the reflector includes a polymeric material having a reflective surface configured to reflect electromagnetic radiation toward the emitter when the reflective surface is dry.

In accordance with another embodiment of the present invention, the medical device further includes wherein the emitter and the sensor are positioned parallel to one another relative to a longitudinal axis (A).

In accordance with another embodiment of the present invention, the medical device further includes a battery and a printed circuit board assembly housed in the needleless connector.

In accordance with another embodiment of the present invention, the medical device further includes a fluid in the reservoir of the cap.

In accordance with an embodiment of the present invention, a system for monitoring a fluid level in a medical device includes a needleless connector including an emitter and a sensor, and a cap removably coupled to the needleless connector, the cap defining a reservoir, the cap including a reflector having a reflective surface, and a medical device management system in communication with the medical device.

In accordance with another embodiment of the present invention, the system further includes a communication network, wherein the medical device management system is in wireless communication with the medical device via the communication network.

In accordance with another embodiment of the present invention, the system is further configured such that the emitter is configured to emit electromagnetic radiation, and the reflective surface is configured to reflect the emitted electromagnetic radiation toward the sensor when the reflective surface is dry.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective schematic of a medical device;

FIG. 2 is a cross-sectional view of the medical device of FIG. 1;

FIG. 3 is an exploded perspective view of a portion of the medical device of FIG. 1;

FIG. 4 is a block diagram of a system for monitoring a fluid level in a medical device;

FIG. 5 is a block diagram of a portion of the system of FIG. 4; and

FIG. 6 is a flow diagram of a method for monitoring a fluid level in a medical device.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

As used herein, the term “computing device” or “computer device” may refer to one or more electronic devices that are configured to directly or indirectly communicate with or over one or more networks. The computing device may be a mobile device, a desktop computer, or the like. Furthermore, the term “computer” may refer to any computing device that includes the necessary components to receive, process, and output data, and normally includes a display, a processor, a memory, an input device, and a network interface. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.).

As used herein, the term “server” may refer to or include one or more processors or computers, storage devices, or similar computer arrangements that are operated by or facilitate communication and processing for multiple parties in a network environment, such as the Internet, although it will be appreciated that communication may be facilitated over one or more public or private network environments and that various other arrangements are possible. Further, multiple computers, e.g., servers, or other computerized devices, such as POS devices, directly or indirectly communicating in the network environment may constitute a “system,” such as a merchant's POS system.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

Operating principals of the disclosed system, method, and medical device may be understood as follows. The emitter 114 and sensor 116 collectively may be electro-optic in that they generally contain an infrared LED and a light receiver. Light from the LED (emitter 114) may be directed into a reflector which forms the tip of the sensor. When no or an inadequate amount of liquid is present, light from the LED is reflected within the reflector to the receiver. This leverages the difference in refractive index between air (~1.0) and 70% IPA (~1.37)—the surfaces can be oriented to effect total internal refraction/reflection for one case (dry), but not the other (wet). When rising liquid immerses the reflector, the light is refracted out into the liquid, leaving little or no light to reach the receiver. Sensing this change, the receiver actuates electronic switching within the unit to operate an external signal or control circuit. Thus, the fluid level in the cap can be poled in real time.

The disclosed system, method, and medical device may be configured to operate with a smart needleless connector. ‘Needleless connector’ may refer to any needle-free luer access port for intravascular med administration, where proper maintenance (cleaning and disinfection) is important to prevent patient infection. ‘Smart’ needleless connectors may include features to determine presence and type of attached disposables—eg a smart connector could determine that a disinfectant cap is attached. The disclosed system, method, and medical device may be useful in detecting defective caps 120 for medical devices 100. The disclosed system, method, and medical device may be useful in detecting defective caps 120 for medical devices 100. Specifically, the disclosed system, method, and medical device may be useful in detecting caps that are dry before first use, eg due to packaging defects or clinician error, neglect, or misuse, or caps that are still ‘wet’ after their first use, e.g. if removed shortly after installation. The disclosed system, method, and medical device may further offer a means to detect, in-situ, exactly when a cap dries out and has maximum anti-microbial effect The disclosed system, method, and medical device provide a solid-state solution with no moving parts that might be prone to failure, dislodgement, etc., and with no new materials exposed to the working fluid. Operation is invisible to the user.

The operating principal of an optical fluid level sensor is well known. At an interface between two materials, the proportion of reflected and transmitted light depends on the angle of incidence and the dissimilarity of refractive indices. Surfaces can be configured such that the amount of light reflected to a sensor changes when one material (working fluid) is replaced with another (air) with a different index of refraction.

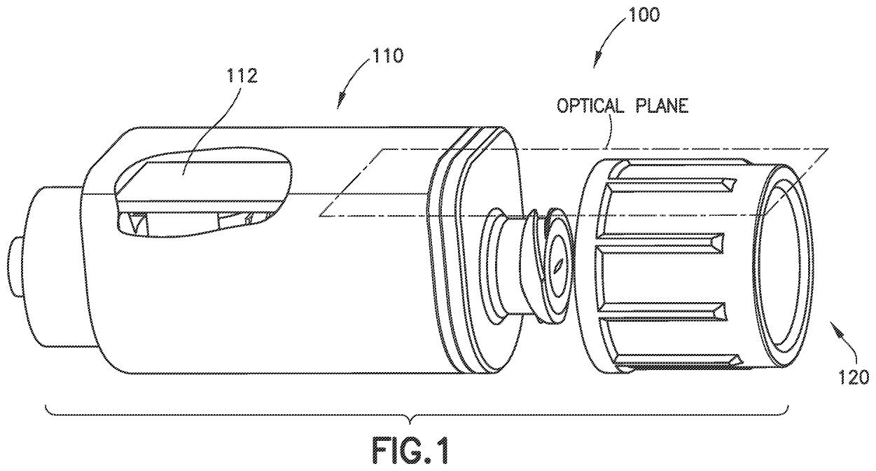

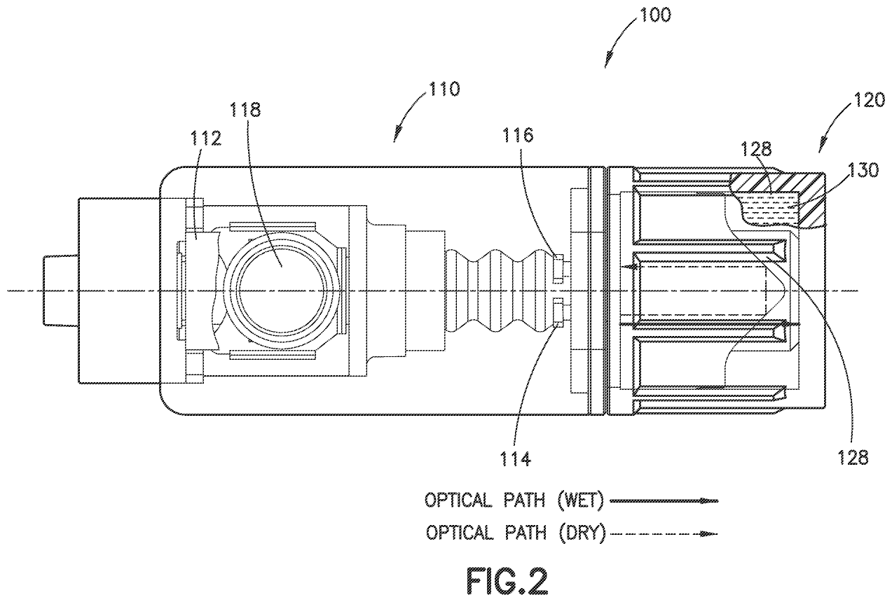

Referring to FIG. 1, disclosed is medical device 100. The medical device 100 includes a needleless connector 110 and a cap 120. Referring to FIG. 2, the needleless connector 110 includes a circuit board 112 coupled with a battery 118, an emitter 114, and a sensor 116. In one example, the emitter 114 and sensor 116 are positioned in the needleless connector 110 such that they are arranged parallel to one another relative to a longitudinal axis A. This disclosed configuration places the emitter 114 and sensor 116 parallel to one another and beneath a flat surface, which may advantageously enhance the cleanability of the needleless connector 110. However, other configurations are contemplated and may be implemented without impacting the functionality of the medical device 100. For example, the emitter 114 and sensor 116 may be positioned in a coaxial arrangement or in an orthogonal arrangement relative to one another. The emitter 114 is configured to emit electromagnetic radiation or light and the sensor 116 is configured to detect the presence of reflected electromagnetic radiation or light emitted from the emitter 114 and reflected back within a detection range of the sensor 116. The sensor 116 may further be in communication, such as wireless communication, with a medical device management system 310, described in further detail below.

The emitter 114 may include one or more sources of electromagnetic radiation. In some non-limiting embodiments, emitter 114 may include a light-emitting diode (LED) configured to emit electromagnetic radiation having a wavelength within the visible light spectrum (e.g. a wavelength in the range of 380 nm to 700 nm). In some non-limiting embodiments, emitter 114 may be an LED configured to emit electromagnetic radiation having a wavelength within the infrared light spectrum (e.g. a wavelength in the range of 800 nm to 1000 nm). In some non-limiting embodiments, emitter 114 may be configured to direct electromagnetic radiation in the direction of a medical device (e.g. a closure device, such as a cap) in proximity to or attached to system for medical device authentication and identification. In some non-limiting embodiments or aspects, emitter 114 may be configured to have at least two states, including a non-activated state in which emitter 114 is not emitting electromagnetic radiation, and an activated state in which emitter 114 is emitting electromagnetic radiation.

In one specific example, the emitter 114 is an infrared (IR) emitter such that it emits electromagnetic radiation having a wavelength within the infrared light spectrum (e.g. a wavelength in the range of 800 nm to 1000 nm) and the sensor 116 is an optical sensor such that it is configured to sense emitted electromagnetic radiation having a wavelength within the infrared light spectrum (e.g. a wavelength in the range of 800 nm to 1000 nm).

In one or more examples, the circuit board 112 of the medical device 100 may be a printed circuit board assembly (PCBA). The circuit board 112 may be associated with the emitter 114 and the sensor 116 so as to provide power via the battery 118.

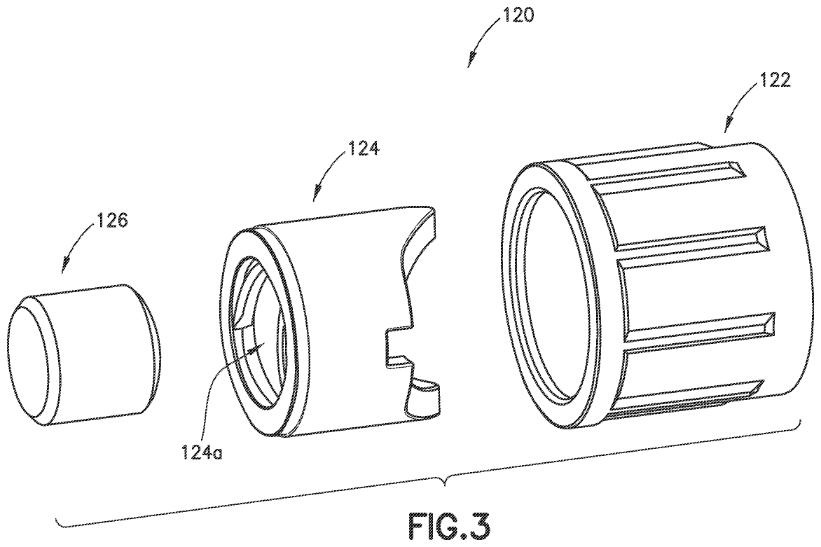

Referring to FIGS. 2 and 3, the cap 120 of the medical device 100 includes a housing 122, a reflector 124, and a foam component 126. The cap 120 defines a reservoir 128 for housing liquid, such as disinfectant. The cap 120 may generally serve as a disinfectant cap that houses a fluid, such as an alcohol or other fluid have disinfectant properties.

In one example, the reflector 124 is a separately molded IR transparent structure having a reflective surface. The reflector 124 may be any material, such as a polymer, having requisite material properties for reflection. In one example, the reflector 124 includes polycarbonate. The reflective surface 124a of the reflector 124 may be configured to reflect or not reflect IR light based upon the amount of fluid 130 in the cap 120. For example, when the surface 124a of the reflector 124 is dry, IR light emitted from the emitter 114 may reflect back to the sensor 116. In the case where the reflective surface 124a of the reflector 124 is wet, IR light emitted from the emitter 114 will not reflect back to the sensor 116. The amount of IR light reflected and detected by the sensor 116 correlate to the fluid level in the cap 120. For example, reflective surface 124a configured to reflect electromagnetic radiation toward the emitter 114 when the reflective surface 124a is dry.

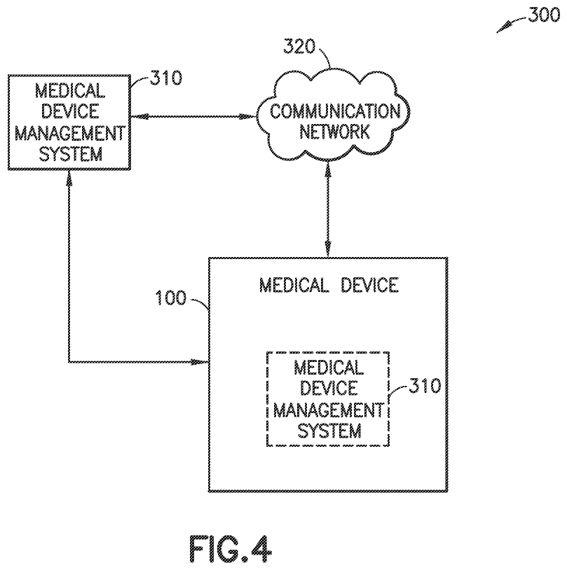

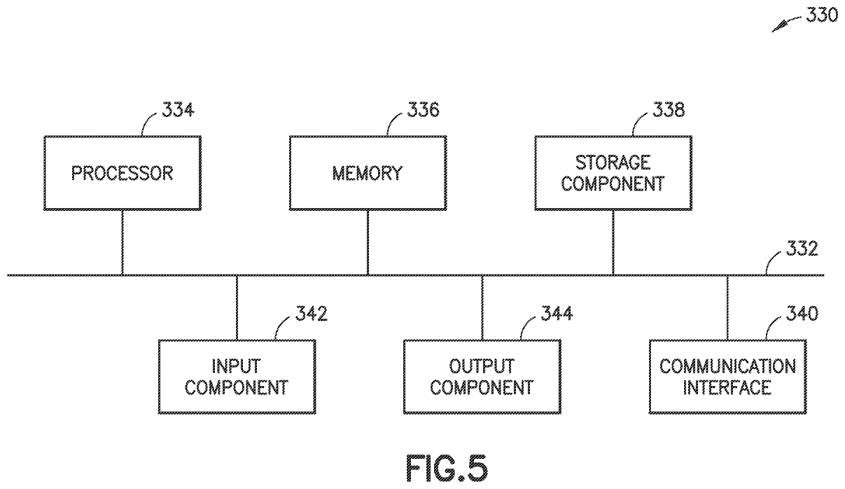

Referring to FIGS. 4 and 5, disclosed is a system 300 for monitoring a fluid level in a medical device 100. The system 300 includes the medical device 100 as shown and described herein. The system 300 further includes a medical device management system 310 and a communication network 320. The medical device management system 310 may interconnect (e.g., establish a connection to communicate, and/or the like) with the medical device 100 via wired connections, wireless connections, or a combination of wired and wireless connections. The medical device management system 310 may interconnect with sensor 116 of the medical device 100 via wired connections, wireless connections, or a combination of wired and wireless connections.

The medical device management system 310 may include one or more devices configured to be in communication with the sensor 116 via the communication network 320. For example, the medical device management system 310 may include a server (e.g., a cloud server), a group of servers, a computing device 330, such as a mobile device (e.g., a smartphone) a tablet computer, a laptop computer, a desktop computer, and/or the like. In some non-limiting embodiments, the medical device management system 310 may be configured to be in communication with the sensor 116 via direct communication connections (e.g., a communication connection that is independent of a communication network 320), such as a short-range wireless communication connection (e.g., a near-field communication (NFC) communication connection, a radio frequency identification (RFID) communication connection, a Bluetooth® communication connection, an infrared communication connection, etc.) or a wired communication connection (e.g., a connection that uses a cable and universal serial bus (USB) communication protocol). In some non-limiting embodiments, the medical device management system 310 may be configured to determine a characteristic of the medical device 100, such as fluid level in the cap 120, based on data 150 received from the sensor 116.

Referring to FIG. 5, the computing device 330 of the medical device management system 310 may include a bus 332, a processor 334, a memory 336, a storage component 338, an input component 342, an output component 344, and a communication interface 340.

Bus 332 may include a component that permits communication among the components of computing device 330. In some non-limiting embodiments, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function.

Memory 336 may include random access memory (RAM), read-only memory (ROM), and/or another type of dynamic or static storage device (e.g., flash memory, magnetic memory, optical memory, etc.) that stores information and/or instructions for use by processor 204.

Storage component 338 may store information and/or software related to the operation and use of computing device 330. For example, storage component 338 may include a hard disk (e.g., a magnetic disk, an optical disk, a magneto-optic disk, a solid-state disk, etc.), a compact disc (CD), a digital versatile disc (DVD), a floppy disk, a cartridge, a magnetic tape, and/or another type of computer-readable medium, along with a corresponding drive.

Input component 342 may include a component that permits computing device 330 to receive information, such as via user input (e.g., a touchscreen display, a keyboard, a keypad, a mouse, a button, a switch, a microphone, a camera, etc.). Additionally or alternatively, input component 342 may include a sensor for sensing information (e.g., a global positioning system (GPS) component, an accelerometer, a gyroscope, an actuator, etc.).

Output component 344 may include a component that provides output information from computing device 330 (e.g., a display, a speaker, one or more light-emitting diodes (LEDs), etc.).

Communication interface 340 may include a transceiver-like component (e.g., a transceiver, a separate receiver and transmitter, etc.) that enables computing device 330 to communicate with other devices, such as via a wired connection, a wireless connection, or a combination of wired and wireless connections. Communication interface 340 may permit computing device 330 to receive information from another device and/or provide information to another device. For example, communication interface 340 may include an Ethernet interface, an optical interface, a coaxial interface, an infrared interface, a radio frequency (RF) interface, a universal serial bus (USB) interface, a Wi-Fi® interface, a cellular network interface, and/or the like.

Computing device 330 may perform one or more processes described herein. Computing device 330 may perform these processes based on processor 334 executing software instructions stored by a computer-readable medium, such as memory 336 and/or storage component 338. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 336 and/or storage component 338 from another computer-readable medium or from another device via communication interface 340. When executed, software instructions stored in memory 336 and/or storage component 338 may cause processor 334 to perform one or more processes described herein. Additionally or alternatively, hardwired circuitry may be used in place of or in combination with software instructions to perform one or more processes described herein. Thus, embodiments described herein are not limited to any specific combination of hardware circuitry and software.

Memory 336 and/or storage component 338 may include data storage or one or more data structures (e.g., a database, and/or the like). Computing device 330 may be capable of receiving information from, storing information in, communicating information to, or searching information stored in the data storage or one or more data structures in memory 336 and/or storage component 338. For example, the information may include data associated with a real-time mobile device application profile, data associated with a historical mobile device application profile, input data, output data, transaction data, account data, or any combination thereof.

The communication network 320 may include one or more wired and/or wireless networks. For example, the communication network 320 may include a cellular network (e.g., a long-term evolution (LTE) network, a third generation (3G) network, a fourth generation (4G) network, a fifth generation (5G), network a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of some or all of these or other types of networks.

The disclosed components of the system 300 are configured to work together to collect data 150 about the amount of fluid 130 present in the cap 120. Upon collection, the data 150 may then be transmitted to the computing device 320 of the medical device management system 310 for further analysis and for generating an alert should the cap 120 not have sufficient fluid 130 to properly function. Accordingly, the disclosed system 300 may be used to perform the method 200 as described below.

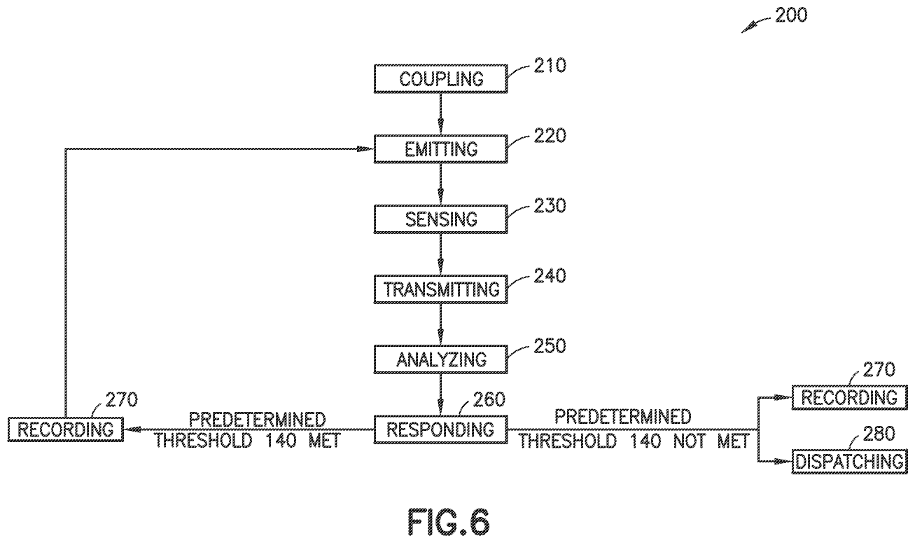

Referring to FIG. 6, disclosed is a method 200 for monitoring a fluid level in a medical device 100. The method 200 may utilize the medical device 100 and system 300 and disclosed herein. The method 200 is useful for monitoring a fluid level in a medical device 100 in that the amount of reflected electromagnetic radiation correlates to an amount of fluid present in the cap 120. If the amount of reflected electromagnetic radiation meets the predetermined threshold 140, the cap 120 has enough fluid 130 for proper functioning. Specifically, the needleless connector 110 has an emitter 114 configured to emit electromagnetic radiation and a sensor 116, and the cap 120 has a reflector 124 having a reflective surface 124a. The reflective surface 124a is configured to reflect the emitted electromagnetic radiation toward the sensor 116 when the reflective surface 124a is dry, which in turn is analyzed to determine and monitor the amount of fluid in the cap 120.

Referring to FIG. 6, the method 200 may include coupling 210 a cap 120 to a needleless connector 110 prior to emitting 220 electromagnetic radiation. Upon the coupling, the medical device management system 310 may recognize that the medical device 100 is assembled and may thus initiate further monitoring methodology to track fluid level in the medical device 100.

The method 200 includes emitting 220 electromagnetic radiation from a first portion of the medical device 100, such as a needleless connector 110, toward a second portion of the medical device 100, such as a cap 120. In one example, the emitting 220 comprises emitting electromagnetic radiation having a wavelength in the range of 800 nm to 1000 nm from an emitter 114.

Still referring to FIG. 6, the method 200 further includes sensing 230 an amount of reflected electromagnetic radiation reflected back toward the first portion of the medical device 100, or needleless connector 110.

Still referring to FIG. 6, the method 200 further includes transmitting 240 a value of the amount of reflected electromagnetic radiation to a medical device management system 310. In one example, the transmitting 240 is performed wirelessly.

The method 200 further includes analyzing 250 the value of the amount of reflected electromagnetic radiation to determine if the amount of reflected electromagnetic radiation meets a predetermined threshold 140. The analyzing 250 may be performed by a medical device management system 310.

The predetermined threshold 140 is a value of the sensed amount of reflected electromagnetic radiation correlating to an amount of fluid 130 in the cap 120. For example, the less amount of reflected electromagnetic radiation, the more fluid 130 is present in the cap 120. Thus, if the predetermined threshold 140 is met, meaning an amount of reflected electromagnetic radiation is at or below the value of the predetermined threshold 140, the medical device management system 310 will recognize that a sufficient amount of fluid 130 is present in the cap 120 and will in turn not prompt a further response indicating that the cap 120 requires attention.

The method 200 further includes responding 260 based upon the analyzing 250. The responding 260 may be based upon the amount of reflected electromagnetic radiation reflected back toward the needleless connector 110. If the amount of reflected electromagnetic radiation reflected back toward the needleless connector 110 meets the predetermined threshold 140, the responding 260 includes recording 270 environmental conditions present at the time of the sensing 230. The environmental conditions present may include the time upon which the sensing 230 occurred and the amount of reflected electromagnetic radiation reflected back toward the first portion of the medical device 100 or needleless connector 110 to correlate to an amount of fluid 130 present in the second portion of the medical device 100 or cap 120. These environmental conditions can then be used for further analytics to monitor fluid level and trends relating to drying rate over time. The medical device management system 310 can further extrapolate a predicted time upon which the fluid 130 will have fully dried and had maximum effect.

The method 200 may further include, after a predetermined period of time T after the recording 270, repeating the emitting 220, sensing 230, transmitting 240, analyzing 250, and responding 260. In one example, the predetermined period of time T is between 2 and 120 seconds. This method 200 may be repeated as many times as needed until the amount of fluid 130 in the cap 120 has dropped to a level such that the amount of reflected electromagnetic radiation reflected back toward the needleless connector 110 is above the predetermined threshold 140.

In the case where the predetermined threshold 140 is not met, meaning more electromagnetic radiation than the value of the predetermined threshold 140 is sensed, the cap 120 does not contain enough fluid 130 and requires attention. Thus, in one example, if the value of the amount of reflected electromagnetic radiation does not meet the predetermined threshold 140, the responding 260 comprises one or more of recording 270 environmental conditions present at the time of the sensing 230 and dispatching 280 an alert. The dispatching 280 an alert may include transmitting a message wirelessly to an external device, such as computing device 330, or back to the medical device 100. In one example, the dispatching 280 an alert includes transmitting a message to the computing device 330 alerting clinicians and/or users of the medical device 100 that the cap 120 is dry, or lacks sufficient fluid 130 to properly function. The alert may include a message that prompts a user to replace the cap, alerts infection control of cap defect and/or misuse or patient tampering, prompts for cap 120 manufacturer code or lot number to assist in QC root cause/defect analysis, or system reports trends/warnings to infection control regarding select lots, clinicians, wards, etc. as appropriate.

The disclosed system 300, method 200, and medical device 100 may be useful in other applications. For example, the disclosed system 300, method 200, and medical device 100 may be implemented for detection of fluid level in other fluid-filled disposables including FLUSH syringes or tubing sets. In the case of FLUSH syringes, the optical pathway could be organized to trigger a ‘low’ reading after partial dispensing. Alternately the pathway could be organized to detect fluid presence at the tip of the syringe, which would help to detect and prevent poor priming. In the case of tubing sets, the optical pathway could be organized to detect bubbles in the line. Alternately the pathway could be organized to detect fluid presence at the tip of the connector, which would help to detect and prevent poor priming.

Although non-limiting embodiments have been described in detail for the purpose of illustration and description, it is to be understood that such detail is solely for that purpose and that embodiments are not limited to the disclosed embodiments, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

1. A method for monitoring a fluid level in a medical device, the method comprising:
emitting electromagnetic radiation from a first portion of the medical device toward a second portion of the medical device, wherein the first portion of the medical device is a needleless connector;
sensing an amount of reflected electromagnetic radiation reflected back toward the needleless connector;
transmitting a value of the amount of reflected electromagnetic radiation to a medical device management system;
analyzing the value of the amount of reflected electromagnetic radiation to determine if the amount of reflected electromagnetic ration meets a predetermined threshold; and
responding based upon the analyzing.

2. The method of claim 1, wherein the second portion of the medical device is a cap.

3. The method of claim 2, further comprising coupling the cap to the needleless connector prior to the emitting.

4. The method of claim 1, wherein the emitting comprises emitting electromagnetic radiation having a wavelength in the range of 800 nm to 1000 nm from an emitter.

5. The method of claim 1, wherein, if the value of the amount of reflected electromagnetic radiation meets the predetermined threshold, the responding comprises recording environmental conditions present at the time of the sensing.

6. The method of claim 5, further comprising, after a predetermined period of time (T) after the recording, repeating the emitting, sensing, transmitting, analyzing, and responding.

7. The method of claim 1, wherein, if the value of the amount of reflected electromagnetic radiation does not meet the predetermined threshold, the responding comprises one or more of recording environmental conditions present at the time of the sensing and dispatching an alert.

8. The method of claim 1, wherein:
the first portion of the medical device comprises an emitter configured to emit electromagnetic radiation and a sensor; and
the second portion of the medical device comprises a reflector having a reflective surface, the reflective surface; configured to reflect the emitted electromagnetic radiation toward the sensor when the reflective surface is dry.

9. The method of claim 1, wherein the amount of reflected electromagnetic radiation correlates to an amount of fluid present in the cap.

10. The method of claim 1, wherein the transmitting is performed wirelessly.

11. The method of claim 1, wherein the analyzing performed by a medical device management system.

12. A medical device comprising:
a needleless connector housing an emitter and a sensor; and
a cap removably coupled to the needleless connector, the cap defining a reservoir, the cap comprising a reflector, wherein the reflector reflects emitted electromagnetic radiation toward the sensor when the reflective sensor is dry, and wherein an amount of reflected electromagnetic radiation correlates to an amount of fluid present in the cap.

13. The medical device of claim 12, wherein the sensor is in communication with a medical device management system.

14. The medical device of claim 12, wherein the emitter includes one or more sources of electromagnetic radiation configured to emit electromagnetic radiation having a wavelength in the range of 800 nm to 1000 nm.

15. The medical device of claim 12, wherein the reflector comprises a polymeric material having a reflective surface configured to reflect electromagnetic radiation toward the emitter when the reflective surface is dry.

16. The medical device of claim 12, wherein the emitter and the sensor are positioned parallel to one another relative to a longitudinal axis (A).

17. The medical device of claim 12, further comprising a battery and a printed circuit board assembly housed in the needleless connector.

18. The medical device of claim 12, further comprising a fluid in the reservoir of the cap.

19. A system for monitoring a fluid level in a medical device, the system comprising:
a medical device comprising:
a needleless connector comprising an emitter and a sensor; and
a cap removably coupled to the needleless connector, the cap defining a reservoir, the cap comprising a reflector having a reflective surface; and
a medical device management system in communication with the medical device,
wherein the reflector reflects emitted electromagnetic radiation from the emitter toward the sensor when the reflective sensor is dry, and
wherein an amount of reflected electromagnetic radiation correlates to an amount of fluid present in the cap.

20. The system of claim 19, further comprising a communication network, wherein the medical device management system is in wireless communication with the medical device via the communication network.

21. (canceled)
